(19) United States
(12) Patent Application Publication
(10) Pub. No.: US 2015/0313525 A1
(43) Pub. Date: Nov. 5, 2015

(54) SAFETY ASSEMBLY FOR MEDICAL TECHNOLOGY

(71) Applicant: GREINER BIO-ONE GMBH, Kremsmünster (AT)

(72) Inventors: Franz EBETSBERGER, Kremsmuenster (AT); Georg KOFLER, Inzersdorf (AT)

(73) Assignee: Greiner Bio-One GmbH, Kremsmuenster (AT)

(21) Appl. No.: 14/648,178

(22) PCT Filed: Nov. 29, 2013
(86) PCT No.: PCT/AT2013/050230

§ 371 Date: May 28, 2015

(30) Foreign Application Priority Data
Nov. 29, 2012 (AT) A-50549/2012

(51) Int. Cl. A61B 5/15

(52) U.S. Cl. CPC A61B 5/150603; A61B 5/150595; A61B 5/150664

(57) ABSTRACT

The invention relates to a safety assembly for medical technology, which comprises a handling device having a main part and a holding attachment and a longitudinal axis extending between a distal and a proximal end. A protective device is moreover provided, having a base part and a protective element connected pivotally thereto, wherein the base part is retained on the holding attachment of the handling device so as to rotate about the longitudinal axis. A locking device having first and second detent elements defines a relative position of the protective device with respect to the handling device. The at least first detent element provided on the handling device is arranged spaced from the holding attachment in the radial direction with respect thereto and spaced from the annular base part on the side facing away from the longitudinal axis. The at least second detent element provided on the base part of the protective device is arranged or formed on the base part on the side thereof facing away from the longitudinal axis. The base part is mounted on the holding attachment of the handling device so as to rotate freely about the longitudinal axis.

The invention relates to a safety assembly in the field of medical technology for taking blood samples, as described in claim 1.

DE 10 2005 054 989 A1 describes a device for taking blood samples, having a needle holder which can be attached to its rear end opposite a sample tube, and a cannula and a needle guard covering the cannula after it has been used are attached to its front end. The needle guard is designed with a cover portion which can be pivoted from a base position into a position over the cannula. The pivotable cover portion is articulatingly connected to a fixing portion and the fixing portion is held locked on a locking shoulder of the needle holder. Mutually facing surfaces of the fixing portion of the needle guard and a pin-type end of the needle holder are disposed between these detent elements. The fixing portion of the needle guard has a central orifice with fluting provided on the internal face.

This fluting co-operates with at least one web of the needle holder lying opposite in a complementary arrangement. This enables a relative positioning of the needle guard to be defined with respect to the needle holder. The disadvantage of this system is that every relative movement between the fixing portion and the pin-type end of the needle holder causes a widening of the seating and this can lead to faults during operation in some circumstances, such as jamming or undesired detachment.

A handling device, a puncture element that can be coupled with it and a pivotable protective device providing a cover after use are known from WO 2004/066840 A1. A positioning device is provided between retaining elements of the protective device and handling device, which defines a pivoting plane of a protective element of the protective device in relation to the position of the handling device when the retaining elements are interconnected. Coupling elements of a coupling device provided between the handling device and puncture element are configured in relation to one another in such a way that when the two coupling elements are in the fully coupled position, a shorter orifice axis of an orifice at the proximal end of the puncture element extends approximately parallel with the pivoting plane or extends in the pivoting plane. The disadvantage of this is the rigid positioning of the pivotable protective device relative to the handling device. —GB 2 277 685 A describes a safety assembly for taking blood samples, having a handling device with a main part of a hollow design which has a distal end having a holding attachment and a proximal end of an open design. A protective device comprises an annular base part and a protective element pivotably connected to the base part which can be pivoted from a released position into a guard position. A needle assembly attached to the handling device can thus be at least partially covered in the guard position. Detent elements extend out from the external surface of the main part which locate in detent recesses in the annular base part in a locked position. When the base part is locked onto the main part, this locked position can only be released by a rotating movement about the longitudinal axis in the opposite direction to enable locking again in another locked position. The base part cannot be freely rotated about the holding attachment whilst simultaneously locking into different locked positions with this system.

U.S. Pat. No. 5,139,489 A discloses a housing holder for accommodating a holder tube which, on a side facing away from the interior, has an extension with an external and an internal thread. A base part of the pivotable protective device has an internal thread by means of which it can be screwed to the external thread of the extension. At its center, this base part also has an orifice which, in terms of its position, coincides with the internal thread disposed in the extension. The needle assembly can be screwed into the internal thread of the extension of the housing holder. To this end, the guard housing can be pivoted about a pivot axis away from the longitudinal axis extending through the housing holder to enable this screwing operation. An exactly definable orientation of the needle end relative to the pivoting plane of the guard housing cannot always be guaranteed.

EP 0 812 597 A2 discloses various designs of a protective device for a needle assembly where either the base part of the protective device is retained on the holder so that it can be pivoted about the longitudinal axis thereof by means of the needle assembly or the base part of the protective element is attached to the needle assembly or is connected to it. The resultant unit can be connected via a standard connection to a syringe housing or a housing holder for blood sample tubes.

U.S. Pat. No. 5,277,311 A and U.S. Pat. No. 5,312,369 A respectively disclose protective devices for needle assemblies, where the base body of the pivotable protective element of the protective device is disposed on the extension of the housing holder or handling device so as to be rotatable or pivotable. In addition, friction-enhancing components may be provided between the extension and the base part in order to set the force which has to be applied in order to turn the base part of the protective device round the extension relative to the longitudinal axis. The protective element can be pivoted via deformable parts of joints from a position in which the needle assembly is released back into a guarding or covering position.

U.S. Pat. No. 3,658,061 A discloses a medical assembly, for example for taking blood samples, comprising a handling device, a puncture element retained on it or inserted in it and a pivotable device for protecting at least a part-region of the puncture element. The protective device is necessarily detachable but is fixedly retained on the handling device, and a flexible part between the protective element and the retaining element of the protective device acts as a joint arrangement. The protective device can be pivoted from a first positioning releasing the puncture element into a second position covering the puncture element. The protective element has a passage for accommodating a part-region of the puncture element and this passage is designed so that the protective element is held on the puncture element in the guard position by means of a friction fit or clamping action.

Another protective element is known from U.S. Pat. No. 4,664,259 A, in which in the region where the protective element sits in the folded-in position, in other words the guard position, a catch element is provided for the protective element by means of which the protective element can be held locked on the puncture element, in particular the hollow needle in the guard position, to prevent or avoid an unintentional release of the proximal end of the puncture element again.

EP 0 626 924 B1 discloses another protective device for a medical assembly, where the protective element is fitted on the handling device so that it has to be detached. In this instance, the retaining element of the protective device is retained on the handling device in a fixed arrangement in the direction of the longitudinal axis, in other words in the axial direction thereof, but the retaining element can be rotated together with the protective element about the longitudinal axis.

Other pivotable needle protective devices are known from EP 0 702 973 B1 respectively EP 0 885 621 B1, where the retaining element for the protective element of the protective device is retained on the puncture element in a region thereof facing away from the handling device in each case. This results in a unit which enables the puncture element to be inserted in the handling device, usually together with the protective element. Guard devices of similar designs are also known from U.S. Pat. No. 6,436,086 B1, U.S. Pat. No. 6,440,104 B1, US 2002/0151852 A1, US 2002/0151853 A1, US 2002/0156425 A1, US 2002/0156427 A1, US 2002/0161336 A1 and US 2002/0193744 A1.

The underlying objective of this invention is to propose a safety assembly for medical technology which offers perfectly reliable, freely rotatable mounting of the protective device on the handling device whilst nevertheless enabling the protective device to be positioned and locked relative to the handling device.

This objective is achieved by the invention due to the characterizing features of claim 1. The advantage obtained as a result of the characterizing features of claim 1 resides in the fact that the mounting or retaining means for the base part of the protective device on the holding attachment of the handling device is spatially separate from the locking device between these components. This spatial separation of the mounting point of the locking device means that every unit can be individually and exactly designed, thereby providing an even more reliable function in terms of the relative positioning operations. For example, depending on the material used for the protective device and handling device, the bearing seat can be designed to suit the usage conditions accordingly to enable a perfect displacement and pivoting movement on the holding attachment about the longitudinal axis. Apart from this, the locking force between the detent elements of the locking device can be set without affecting the bearing seat. Furthermore, mutual jamming between the components which are still able to rotate relative to one another can be prevented. In addition, the retaining force applied in the locked position on the one hand and the force needed in rotation to overcome the locking force generated between the detent elements on the other hand can be fixed. The ability to rotate freely on the holding attachment remains unaffected by this. This being the case, the protective device together with its base part can be rotated about the holding attachment providing a mount for the base part depending on the number and disposition of detent elements between the individually provided locking positions around the base part.

Another embodiment defined in claim 2 is also of advantage because several locking positions between the protective device and handling device can be defined around the circumference, thereby making it possible to adapt to the personal requirements of the medical personnel in terms of correct usage.

A design defined in claim 3 is also of advantage because it enables a stable rim to be created on the external circumferential region of the end wall, which helps to impart additional strength to the handling device. Furthermore, this enables an unimpeded movement to be obtained so that mutual jamming by other protruding parts can be avoided.

As a result of the embodiment defined in claim 4, even better protection can be obtained for the locking device and the base part of the protective device retained on the holding attachment. Tampering can therefore also be made more difficult, thereby enabling the overall safety of the safety assembly to be increased. Furthermore, depending on the wall thickness selected for the collar, the retaining force as well as the force needed for the rotating movement can be predefined.

Based on another embodiment defined in claim 5, a defined support plane is created for the base part in the region of the holding attachment, as a result of which an exact guiding action and associated mount are obtained for the protective device for its movement about the longitudinal axis. This also results in a compact design of the handling device so that the safety assembly requires the minimum amount of material.

Another embodiment defined in claim 6 is of advantage because regardless of the fact that the base part is mounted on the holding attachment or retaining part, a perfect locking action and hence fixed, predefined relative positioning of the protective element can be obtained with respect to the handling device. Furthermore, however, a certain pressing force of the base part on the holding attachment is also obtained.

The advantage of the design defined in claim 7 is that a symmetrical locking arrangement between the base part and handling device is obtained. Moreover, this also results in a certain degree of centering as well as providing a mount on the holding attachment. The diametrically opposite, slot-shaped recesses also impart a certain degree of additional flexibility or elasticity to the base part to enable the fitting movement on the holding attachment on the one hand and to permit a certain degree of deformation of the base part for the movement about the longitudinal axis.

The objective of the invention can also be achieved independently on the basis of the characterizing features defined in claim 8. The advantages obtained as a result of this combination of features reside in the fact that again, mounting or retaining means of the base part of the protective device on the holding attachment of the handling device is spatially separate from the locking device between these components. This spatial separation of the mounting point of the locking device means that every unit can be individually and exactly designed, thereby providing an even more accurate function in terms of the relative positioning operations. For example, depending on the material used for the protective device and handling device, the bearing seat can be designed to suit the usage conditions accordingly to enable a perfect displacement and pivoting movement on the holding attachment about the longitudinal axis. Apart from this, the locking force between the detent elements of the locking device can be set due to the axial height of the detent elements without affecting the bearing seat. Furthermore, mutual jamming between the components which are still able to rotate relative to one another can be prevented. In addition, the retaining force applied in the locked position on the one hand and the force needed in rotation to overcome the locking force generated between the detent elements on the other hand can be fixed.

As a result of the design defined in claim 9, a locking force can be generated between the end wall of the handling device and the base part, based on a shearing force between the base part and end wall of the handling device. Furthermore, this results in a component that is simple to manufacture, is easy to remove from the mold and also affords a reliable lock between the co-operating components.

Also of advantage is an embodiment as defined in claim 10 because depending on the three-dimensional shape selected for the detent element, the locking force or locking action on the one hand and the force needed to effect the displacement on the other hand can be easily adapted to different conditions of usage.

Yet another embodiment defined in claim 11 is of advantage because several locking positions between the protective device and handling device can be defined around the circumference, thereby enabling improved individual adaptation to the personal requirements of the medical personnel in terms of correct usage.

In this respect, an embodiment as defined in claim 12 has proved to be of advantage because in the locked and coupled position, tampering is prevented by the mutual covering position between the detent elements. However, externally acting influences which might detrimentally affect the locking device can also be largely ruled out.

Based on another advantageous embodiment defined in claim 13, a symmetrical locking action can be obtained between the base part and handling device. Moreover, this also results in a certain degree of centering as well as providing a mount on the holding attachment. The diametrically opposite, slot-shaped recesses also impart a certain degree of additional flexibility or elasticity to the base part to enable the fitting movement on the holding attachment on the one hand and to permit a certain degree of deformation of the base part for the movement about the longitudinal axis.

An embodiment as defined in claim 14 is also of advantage because when the needle assembly is inserted, a more exact bearing and retaining hold can be obtained for the base part on the holding attachment. Furthermore, however, the protective device is prevented from being removed in all operating situations so that it is prevented from being used again if the safety assembly has already been used.

Finally, however, based on a design described in claim 15, a certain degree of axial movement of the base part on the holding attachment is possible but a predefined retaining or locking force is always generated by the spring element in the locked position. However, the force which needs to be applied in order to move the base part about the longitudinal axis can then be reduced because the spring element permits a certain degree of axial longitudinal displacement of the base part on the holding attachment, and once another preselected locking position is reached, a retaining force sufficient to position the protective device with respect to the handling device is generated again.

To provide a clearer understanding, the invention will be described in more detail with reference to the appended drawings.

These are highly schematic, simplified diagrams illustrating the following:

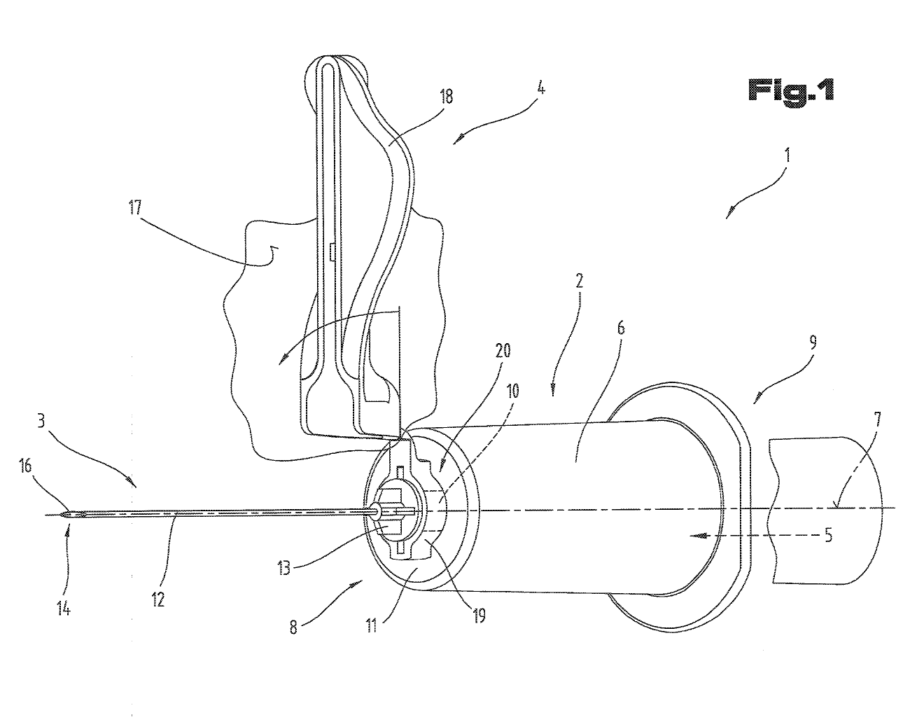

FIG. 1 a diagrammatic illustration of a safety assembly in the fully assembled state;

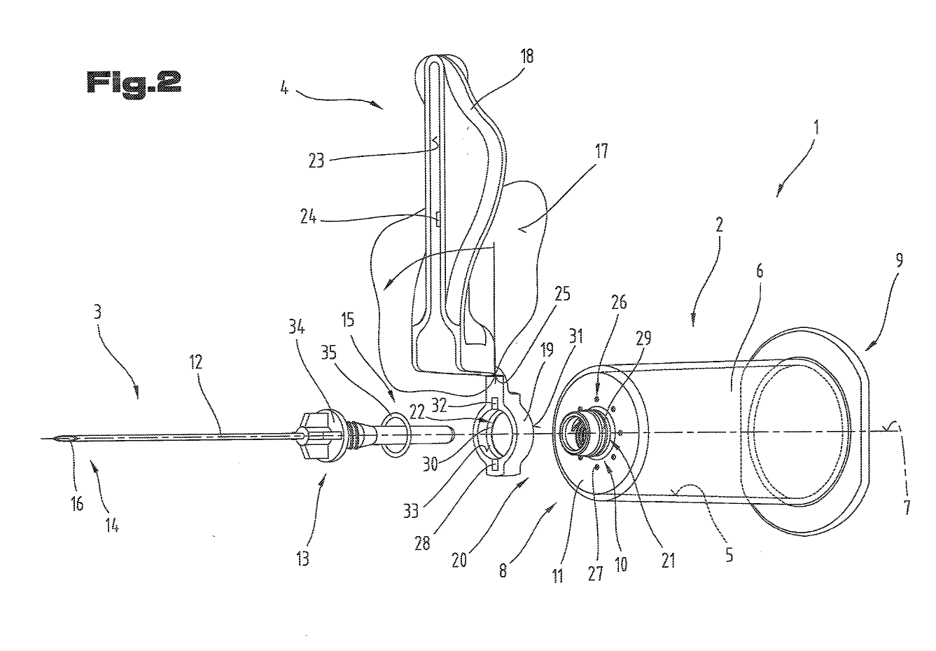

FIG. 2 a diagrammatic illustration of the safety assembly shown in FIG. 1 with the individual components in a position spaced apart from one another;

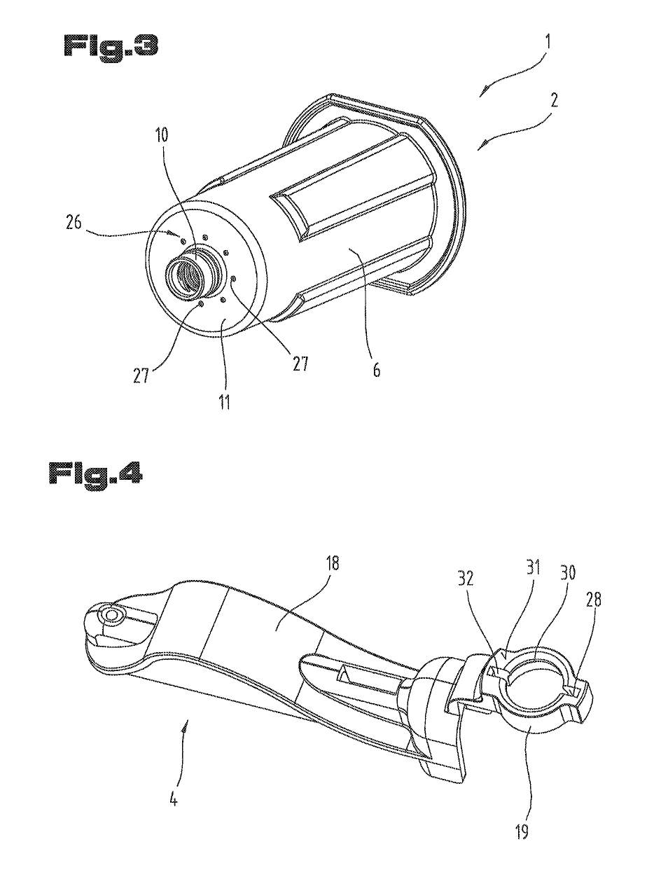

FIG. 3 a diagrammatic illustration of the handling device of the safety assembly shown in FIGS. 1 and 2 with first detent elements disposed thereon;

FIG. 4 a diagrammatic illustration of the protective device of the safety assembly shown in FIGS. 1 and 2 with the second detent elements;

FIG. 5 an axial section of the safety assembly shown in FIGS. 1 to 4 in the region of the locking device;

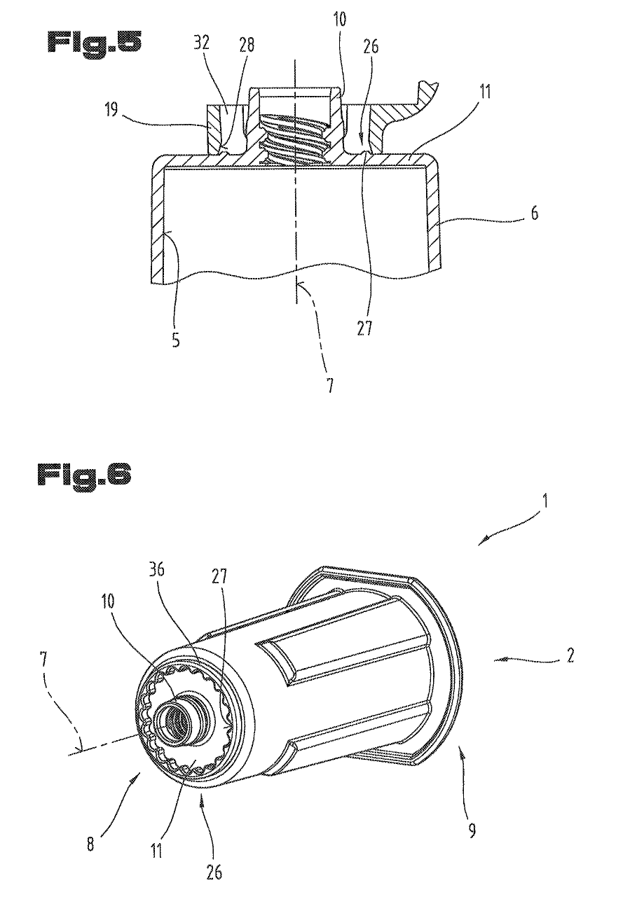

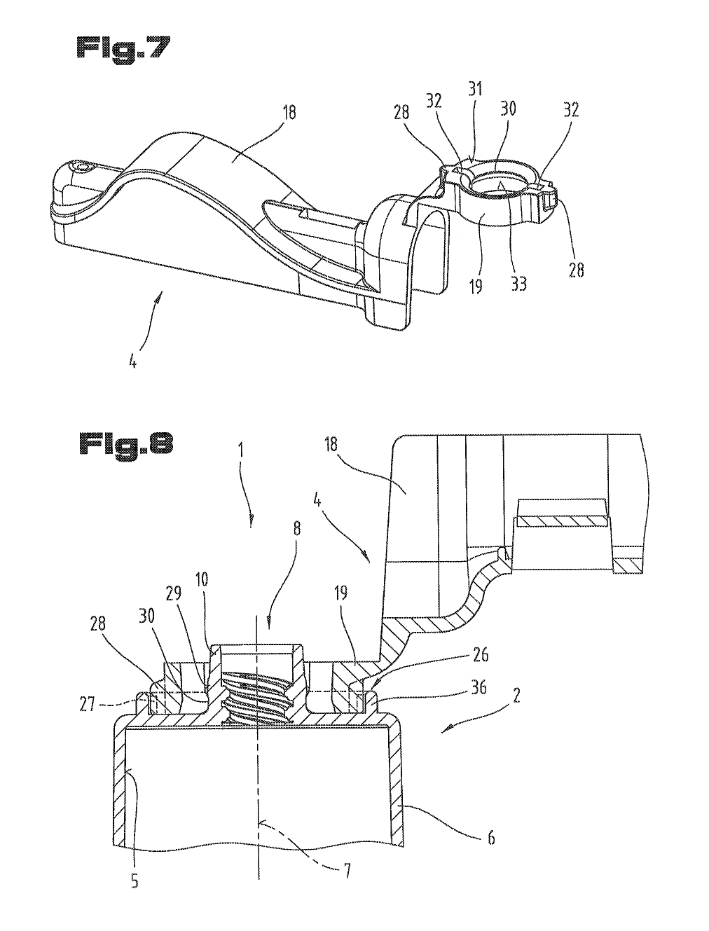

FIG. 6 a diagrammatic illustration of another possible embodiment of the first detent elements on a handling device;

FIG. 7 a diagrammatic illustration of the protective device of the safety assembly for the handling device shown in FIG. 6 with the second detent elements;

FIG. 8 an axial section of the safety assembly illustrated in FIGS. 6 and 7 in the region of the locking device;

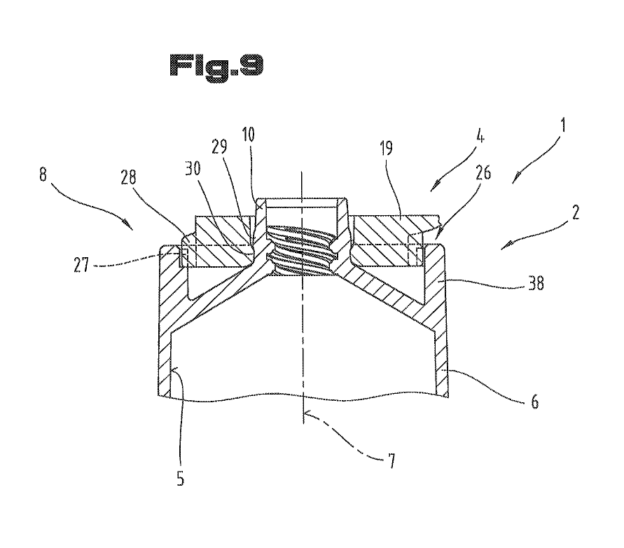

FIG. 9 a diagrammatic illustration of another embodiment of the first detent elements on a handling device.

Firstly, it should be pointed out that the same parts described in the different embodiments are denoted by the same reference numbers and the same component names and the disclosures made throughout the description can be transposed in terms of meaning to same parts bearing the same reference numbers or same component names. Furthermore, the positions chosen for the purposes of the description, such as top, bottom, side, etc., relate to the drawing specifically being described and can be transposed in terms of meaning to a new position when another position is being described.

FIGS. 1 to 5 illustrate a safety assembly 1, in particular for taking blood samples in the field of medical technology, comprising at least one handling device 2, a needle assembly 3 that can be mounted on it as and when necessary, as well as a pivotable protective device 4 for at least a partial region of the needle assembly 3.

The handling device 2 may be provided in the form of a hollow main part 6, for example, forming or surrounding a housing chamber 5, having a distal end 8 and a proximal end 9 spaced apart from one another in the direction of a longitudinal axis 7. The proximal end 9 in this instance is open and is used to accommodate at least a part of a container, also referred to as a blood sample tube, which is illustrated on a simplified basis but will not be described in detail.

A holding attachment 10 is provided or disposed in the region of the distal end 8 of the main part 6. The holding attachment 10 may be used and designed to accommodate and hold the needle assembly 3 and/or protective device 4. The distal end 8 is preferably closed in at least certain regions by means of an end wall 11, illustrated here on a simplified basis.

It should generally be pointed out that the term “distal” as used in this application always refers to the part of the safety assembly 1 which, when correctly used by a user (medical personnel), is facing away from him/her and therefore faces the patient. The term “proximal” always refers to the part of the safety assembly 1 which, when correctly used by a user (medical personnel), faces him/her and is therefore facing away from the patient. Accordingly, individual components of the safety assembly 1 respectively have an end facing the distal end 8 or the proximal end 9 as viewed in the direction of the longitudinal axis 7 in each case.

In the embodiment illustrated as an example here, the needle assembly 3 comprises a cannula 12, a retaining part 13 disposed on it which is disposed, as viewed in the direction of the longitudinal axis 7, between a distal cannula end 14 and a proximal cannula end 15 spaced apart therefrom. A continuous orifice between the two cannula ends 14, 15 connects the latter to one another and constitutes a flow passage during use as intended. This permits a flow through the hollow cannula 12.

The holding attachment 10 serves as a coupling element of a coupling device, by means of which the retaining part 13 of the needle assembly 3 can be connected or coupled. In this example of an embodiment, this is done by means of a thread system in a manner that has long been known. In this respect, a multi-start thread may also be used. The holding attachment 10 is of an approximately tubular design and a part of the thread system is disposed on its inside or internal face. Accordingly, the needle assembly 3 can be fitted on the handling device 2 so that it can be detached if necessary.

If the cannula 12 is provided in the form of a double-ended hollow needle—as illustrated in this example of an embodiment—a partial region of the cannula 12—namely its distal cannula end 14—extends out beyond the handling device 2 towards the end remote from the housing chamber 5. As illustrated on a simplified basis and in a manner known per se, the cannula 12 is angled in the region of the distal cannula end 14 forming an orifice 16 which approximately has the shape of an ellipsis or oval due to the angled orientation relative to the longitudinal axis 7. As a result, the orifice 16 has a longer as well as a shorter orifice axis.

The mutually engaging threads of the thread system are provided in the form of an internal thread on the base part 19 and an external thread on the retaining part 13 of the needle assembly 3 which can be moved into engagement with it. The thread system is preferably provided in the form of a two-start thread, thereby making fitting possible and resulting in an associated end position of the orifice 16 alongside the cannula 12, oriented at 180° relative to one another.

The protective device 4 has at least one protective element 18 configured to be pivotable in a pivoting plane 17, which can be pivoted from a first position exposing the partial region of the cannula 12 (released position) into a second position covering the partial region (guard position).

In order to mount the protective device 4 on the handling device 2, it has a base part 19 with an approximately tubular or annular design, by means of which it can be retained on the holding attachment 10 of the handling device 2, optionally in a detachable manner. In conjunction with the base part 19, the holding attachment 10 constitutes a retaining and locking mechanism 20. The base part 19 is also retained on the holding attachment 10 of the handling device 2 so that it is able to rotate about the longitudinal axis 7.

The locking mechanism 20 enables the protective device 4, in particular the base part 19, to be assembled with and hence fitted on the holding attachment 10. However, it could also be used to enable the pivotable protective device 4 to be detached or separated from the handling device 2 if necessary. It is preferable to provide a pre-assembled safety assembly 1, whereby both the protective device 4 and the needle assembly 3 are already assembled with the handling device 2 in the respective position of usage. For safety reasons, these can no longer be separated and once used for the intended purpose and with the protective element 18 disposed in the guard position, the entire safety assembly 1 is disposed of. Accordingly, the co-operating components are configured relative to one another so that a mutual hold is obtained by means of a locked connection but it is still possible for the base part 19 to rotate about the longitudinal axis 7 and hence about the holding attachment 10. Any movement in the axial direction is not possible or is so to only a slight extent.

If the holding attachment 10 on the main part 6 of the handling device 2 is provided in the form of a component based on an approximately tubular design, a central axis of this component may be oriented so that it extends congruently with the longitudinal axis 7. The holding attachment 10 is generally disposed in a fixed arrangement concentric with the longitudinal axis 7 of the main part 6 in the region of the distal end 8 at least partially closed by the end wall 11. This middle or central disposition relative to the main part 6 is used in the case of a double-ended cannula 12, in which case its distal cannula end 14 extends beyond the end wall 11 in the direction remote from the housing chamber 5, as described above, and the proximal cannula end 15, when in a position fitted on the handling device 2, extends in the direction towards the open proximal end 9 into the housing chamber 5. This disposition of the cannula 12 relative to the handling device 2 is known in this form. Although this will not be described in further detail, a flexible protective sleeve may be used to cover the part of the cannula 12 extending into the housing chamber 5 which is then pierced and moved when a flow connection is established with the interior of a sample tube.

The holding attachment 10 projects out from the end wall 11 towards the end remote from the housing chamber 5. The approximately tubular or annular base part 19 locates round the holding attachment 10 in the region of its external surface, in particular in the region of the cylindrical surface, at least in certain regions. Accordingly, an external dimension or an external diameter of the holding attachment 10 is more or less the same as the internal dimension or internal diameter of the base part 19 and, because of the selected dimensions, the base part 19 can be pushed or slid onto the holding attachment 10 in the axial direction. To prevent the two components from being detached once in the located position, the locking mechanism 20 may also comprise yet other mutually locatable locking elements 21, 22, as may best be seen by comparing FIGS. 2 and 5. These two locking elements 21, 22 may be disposed around at least certain regions of the circumference of the holding attachment 10 or base part 19 on mutually facing sides. However, the holding attachment 10 and the base part 19 may also be of any other shape, e.g. oval, polygonal, etc.

In the embodiment illustrated as an example here, the first locking element 21—see FIG. 2—is disposed on the holding attachment 10, namely the component based on a tubular design, in the form of a bead 29 projecting radially out from the holding attachment 10 towards the side remote from the longitudinal axis 7, although it may also extend continuously around the circumference of the holding attachment 10, at least in certain regions.

The other locking element 22 is disposed on the tubular or annular base part 19 on the side facing the longitudinal axis 7, projecting out from the internal surface in the form of another bead 30, as may best be seen from FIGS. 2 and 5. This bead 30 constituting the other locking element 22 may in turn extend around at least certain regions of the circumference of the base part 19 and, in the locked position, the other locking element 22 locates behind the first locking element 21 on the holding attachment 10 disposed on the side facing the end wall 11.

As a result of the embodiment described here, which represents but one of a number of possible designs for the co-operating locking elements 21, 22, when the base part 19 and holding attachment 10 are connected or engaged, an inadvertent movement of the two components relative to one another in the direction of the longitudinal axis 7 is prevented. Based on the design of the co-operating locking elements 21, 22 described above, the handling device 2 is held in a stationary arrangement but the protective device 4 is able to rotate or turn about the longitudinal axis 7 relative to the handling device 2. However, the locking element 21 may also be provided in the form of one or more groove-shaped recesses on the holding attachment 10, in which case the other locking element 22 is provided in the form of one or more projections on the base part 19 co-operating or engaging with it/them.

As illustrated on a simplified basis in FIG. 2, the protective element 18 has a passage 23 for accommodating at least a partial region of the cannula 12 in the region of its distal cannula end 14. In order to prevent the protective element 18 from inadvertently pivoting back out of the guard position, at least one retaining element 24 is provided in the region of the passage 23 to provide a locked hold on the partial region of the cannula 12 in the guard position, in a manner already known from the prior art.

To enable the pivoting movement of the protective element 18 relative to the base part 19, an elastically deformable web may be provided between these components. To obtain a more exact pivoting movement and a more exact position of the protective element 18 in the guard position covering the partial region of the cannula 12, it is of advantage if the protective element 18 is able to pivot on a pivot axis 25 shaft oriented perpendicular to the pivoting plane 17, in which case this pivot axis 25 is preferably obtained by provided a film hinge.

Furthermore, a locking device 26 with at least a first detent element 27 is provided on the handling device 2 as well as at least a second detent element 28 on the base part 19 of the protective device 4, and when the first and second detent elements 27, 28 are engaged, a position of the protective device 4, in particular its protective element 18, relative to the handling device 2 is fixed. This is the position of the protective element 18 in relation to the angular position about the longitudinal axis 7.

Due to the detent elements 27, 28 co-operating with one another in the locked position, eye contact with the approximately elliptical or oval orifice 16 can always be adjusted based on an appropriate orientation, such as is necessary when the safety assembly 1 is being used for taking samples of body fluids and/or when administering fluid substances to the body for medical purposes. The handling device 2, protective device 4 and the assembled medical safety assembly 1 are usually used in the field of medicine for taking blood samples.

Accordingly, the user of the safety assembly 1 can adapt and adjust the protective element 18 of the protective device 4 individually to the conditions of usage and his/her personal requirements. When taking a blood sample, for example, when used in accordance with the instructions, the protective element 18 is moved so that it is not facing the patient's arm and is therefore not a hindrance when taking a sample. It is therefore always possible to view or see the orifice 16 as is necessary when puncturing a body part such as a vein or artery, for example, to ensure that the puncturing operation is performed correctly.

The advantage of this medical safety assembly 1 is that the protective device 4 can already be pre-fitted on the handling device 2 independently of the needle assembly 3 and the needle assembly 3 does not have to be fitted on the handling device 2 until such time as needed for use—when it can be screwed on in this case. Alternatively, however, the needle assembly 3 may also be pre-assembled, in which case it is already attached to the handling device 2. This obviates the need for other connection operations, thereby avoiding the risk of injury due to puncturing. Irrespective of this, however, it would also be possible to connect the cannula 12 directly to the main part 6, in particular its holding attachment 10. This could be done by gluing, welding or other similar joining operations, for example.

When used correctly, the protective element 18 is disposed to the side of the handling device 2 without assuming a position that will either cause an obstruction between the patient's arm and the handling device 2 or obstruct eye contact with the orifice 16 of the cannula 12 facing the user, for example a doctor.

The handling device 2, protective device 4 and retaining part 13 for the cannula 12 described here are usually made from a plastic, and the handling device 2 is preferably of a transparent or see-through design, making it possible to see into the interior.

As described above, the base part 19 of the protective device 4 is mounted or retained on the holding attachment 10 of the handling device 2 so as to be rotatable. To prevent a freely rotating action and enable pre-setting and individual adjustment of the positioning of the protective element 18 relative to the oblique end of the cannula 12, namely the orifice 16, the locking device 26 is provided. In the embodiment illustrated as an example here, the at least first detent element 27 provided on the handling device 2 is disposed on the end wall 11. The at least second detent element 28 provided on the base part 19 of the protective device 4 is disposed in the region of an end face 31 of the base part 19 directed towards the end wall 11. These might be provided in the form of a cut-out, recess or similar. In this particular example of an embodiment, the at least first detent element 27 on the end wall 11 extends out from the latter in the axial direction. For example, the first detent element 27 may be selected from the group comprising studs, radial teeth, projections, recesses. However, the second detent element 28 may also extend out from the end face 31 in the axial direction and locate in a recess provided in the end wall 11 for this purpose.

To enable several locking positions to be individually set around the circumference, it would also be possible to provide several first detent elements 27 circumferentially distributed on the end wall 11 of the handling device 2.

In order to provide the second detent element 28 on the base part 19 of the protective device 4, at least one recess 32 is provided on the base part 19 in its end face 31 directed towards the end wall 11 in this instance. This recess 32 in the end face 31 therefore constitutes the at least one second detent element 28. In the corresponding locked position, the first detent element 27 is supported on at least one side wall bounding the recess 32 in the circumferential direction.

As illustrated in this example of an embodiment, the base part 19 of the protective device 4 has at least two diametrically opposite recesses 32 in its internal face 33 directed towards its longitudinal axis 7. In this example of an embodiment, these recesses 32 extend continuously in the axial direction between the distal end region and the proximal end region of the base part 19. The recesses 32 may be of a slot-shaped or groove-shaped design, for example.

The fact that the slot-shaped recesses 32 are provided means that a certain amount of additional elasticity of the base part 19 can be achieved during the fitting movement on the holding attachment 10 of the handling device 2. This results in greater flexibility during the pushing-on or fitting operation and once the locking elements 21, 22 described above have been moved into engagement, a sufficiently firm but rotatable seating of the base part 19 on the holding attachment 10 is achieved. The recesses 32 therefore fulfil a dual function and can be moved into a locked connection with the first detent elements 27 on the end wall 11 by an appropriate mutual orientation or angular position relative to one another.

As also illustrated, the needle assembly 3, in particular its retaining part 13, has a neck 34, in particular based on a flange-shaped design, which extends at least partially out from the base part 19 in the radial direction when the needle assembly 3 is in the position coupled with or screwed into the holding attachment 10 of the handling device 2. The neck 34 may also be described as a retaining flange. It prevents the protective device 4 from being removed from the handling device 2. This is of particular importance after use when the protective element 18 is pivoted into the covering guard position over the distal cannula end 14 to be covered and secured by the retaining element 24 to prevent it from being used again. If this were not the case, even though the protective element 18 is held in a latched or locked position on the cannula 12 in the guard position, it would be possible to pull the entire protective device 4 off the handling device 2 in the axial direction. This is prevented due to the fact that the neck 34 is locked onto the base part 19.

By choosing the dimensions of the holding attachment 10, base part 19 and the axial distance between the end wall 11 and contact surface of the neck 34 facing it accordingly, and in conjunction with the engaged locking elements 21, 22, the axial clearance of the base part 19 as well as the engaged detent elements 27, 28 can be influenced.

As also illustrated in the region of the needle assembly 3, a schematically illustrated spring element 35 is provided between the neck 34 and the base part 19, shown on a simplified basis in the form of a circular or toroidal component. The expression spring element 35 in this context should be understood as meaning any component which is compressible at least in the axial direction and builds up or generates a compression force when axially deformed or biased. For example, the spring element 35 might be a flexible ring made from a varied range of materials or alternatively a compression spring, a serrated lock washer, a tooth lock washer or such like. This spring element 35 enables a certain degree of axial play of the base part 19 to be obtained on the holding attachment 10. The spring element 35 then generates an appropriate pressing force in the axial direction towards the base part 19, which is pressed so that its end face 31 sits on the end wall 11. Since the locking device 26 is disposed with its detent elements 27, 28 lying opposite one another in a plane, the force which locks the locking device 26 can also be influenced. The locking force therefore acts in the axial direction between the detent elements 27, 28.

FIGS. 6 to 8 illustrate another embodiment of the safety assembly 1 which may be optionally be construed as an independent solution in its own right, the same reference numbers and component names being used to denote parts that are the same as those described in connection with FIGS. 1 to 5 above. To avoid unnecessary repetition, reference may be made to the detailed description of FIGS. 1 to 5 given above.

This additional option for the design of the locking device 26 illustrated here with its first and second detent elements 27, 28 can in turn also be used to set the relative position of rotation of the protective element 18 of the protective device 4 in the circumferential direction with respect to the longitudinal axis 7. Here too, the illustrated safety assembly 1 comprises the handling device 2 having the hollow main part 6 with the distal end 8 and proximal end 9. In the region of the distal end 8, the end wall 11 may be provided, on which the holding attachment 10 is disposed, projecting in the axial direction. The proximal end 9 is of an open design and is used to accommodate at least a part of a container, which is an evacuated sample tube enabling a body fluid to be contained, for example.

The protective device 4 also has the base part 19 based on a tubular or annular design, to which the protective element 18 is pivotably connected.

By contrast with the design of the locking device 26 described above, in this instance the at least one first detent element 27 provided on the handling device 2 is disposed, as viewed in the radial direction, at a distance apart from the holding attachment 10 on the side facing away from the longitudinal axis 7. In order to provide several differently oriented locking positions around the circumference, several first detent elements 27 may also be provided on the handling device 2 in a circumferentially distributed arrangement. These may be provided in the form of individual, mutually separate detent elements 27 spaced at a distance apart from one another in the circumferential direction.

The or the first detent elements 27 are also provided or disposed on the handling device 2 at a distance apart from the base part 19 of the protective device 4 in the radial direction due to their radial spacing from the holding attachment 10, as may be seen from FIG. 6 in conjunction with FIG. 8. Accordingly, the or the first detent elements 27 are disposed lying opposite the external face of the base part 19 and are therefore directed towards the external circumference of the base part 19. As viewed in the radial direction, the base part 19 is disposed between the holding attachment 10 and the or the first detent elements 27. In spite of the externally disposed locking device 26, the base part 19 is still mounted on the holding attachment with its internal face directed towards the longitudinal axis 7 so as to be freely rotatable.

In the embodiment illustrated as an example here, several of the first detent elements 27 are disposed on or so as to form a circumferentially continuous tubular or annular neck 36. Accordingly, the first detent elements 27 constitute a type of wave-shaped internal toothing. As viewed in the circumferential direction, a differing radial spacing from the longitudinal axis 7 is formed by this waved shape, and the first detent element 27 is formed either by the recess having the bigger radial distance or by the protuberance between the recesses directed towards the longitudinal axis 7.

Not only does the main part 6 of the handling device 2 illustrated here have the end wall 11 oriented perpendicular to the longitudinal axis 7 with the holding attachment 10d disposed thereon and extending out from it in the axial direction on its distal end 8 but also the at least first detent element 27. The orientation and direction in which the locking force acts between the detent elements 27, 28 is therefore in a plane oriented perpendicular to the longitudinal axis 7 in the radial direction with respect to the longitudinal axis 7.

As may be seen more clearly from FIG. 7, the at least second detent element 28 provided on the base part 19 is disposed so that it extends out from the base part 19 in the radial direction. When the protective device 4 is fitted on and coupled with the handling device 2, the other detent element 28 extends into the first detent element 27 provided in the form of a recess in the locked position in this example of an embodiment. The base part 19 is again mounted so that it is able to rotate or turn on the holding attachment 10 of the handling device 2.

The base part 19 of the protective device 4 illustrated here also has the at least two diametrically opposite, slot-shaped recesses 32 on its internal face 33 directed towards the longitudinal axis 7. They interrupt the annular base part 19 in the region of its internal face 33 constituting a circumferentially continuous component. These base part halves disposed in an axial plane symmetrically and in mirror image with respect to one another are connected to one another in the region of the recesses 32 by a web-type component on the side facing away from the longitudinal axis 7 in each case. The or the other detent elements 28 are then disposed or formed on this web-type component. The or the second detent elements 28 are therefore disposed on an external face 37 of the base part 19 facing away from the longitudinal axis 7. Where the recesses 32 are provided, the second detent elements 28 are disposed respectively lying opposite the recesses 32.

Again in this instance, the base part 19 may be retained and locked on the holding attachment 10 by means of the locking mechanism 20 described above co-operating with the first and second locking elements 21, 22, as already described in detail above.

In view of the fact that the locking force to be overcome during the movement about the longitudinal axis 7 acts in the radial direction, the base part 19 can be mounted and retained on the holding attachment 10 so as to rotate with a relatively small axial clearance. The retaining force acting in the axial direction can therefore be expended by the locking mechanism 20 by means of its locking elements 21, 22 and/or via the retaining action of the neck 34 projecting in the radial direction on the retaining part 13 of the needle assembly 3 on the holding attachment 10 and hence the handling device 2.

FIG. 9 illustrates another embodiment of the handling device 2 forming the safety assembly 1 which may be construed as an independent embodiment in its own right, the same reference numbers and component names being used to denote parts that are the same as those used in connection with FIGS. 1 to 8 described above. To avoid unnecessary repetition, reference may be made to the detailed description of FIGS. 1 to 8 above.

The embodiment of the locking device 26 illustrated in this instance is similar to that described above in connection with FIGS. 6 to 8 in terms of design and the way it operates. The protective device 4, in particular the base part 19, may also be of the same design as that described with respect to FIGS. 6 to 8.

The only difference here is the disposition and design of the other detent elements 28 on the handling device 2. The holding attachment 10 in this instance is not provided on an end wall 11 disposed perpendicular to the longitudinal axis 7 but rather is connected thereto by a transition portion opposite the main part 6 tapering towards the distal end 8. To provide and form the other detent elements 28, a collar 38 is provided on the main part 6 in this instance, in the region of its distal end 8, which is disposed approximately concentrically with respect to the holding attachment 10 and extends out from the main part 6 in the axial direction. The collar 38 is spaced in the radial direction with respect to the holding attachment 10 apart therefrom on the side facing away from the longitudinal axis 7. The at least second detent element 28 is disposed or formed on it. The design of the second or other detent element 28 may again be the same as that illustrated and already described above in connection with FIG. 5.

For the sake of good order, finally, it should be pointed out that in order to provide a clearer understanding of the structure of the safety assembly 1, it and its constituent parts are illustrated to a certain extent out of scale and/or on a larger scale and/or on a smaller scale.

The objective underlying the independent inventive solutions may be found in the description.

All the figures relating to ranges of values in the description should be construed as meaning that they include any and all part-ranges, in which case, for example, the range of 1 to 10 should be understood as including all part-ranges starting from the lower limit of 1 to the upper limit of 10, i.e. all part-ranges starting with a lower limit of 1 or more and ending with an upper limit of 10 or less, e.g. 1 to 1.7, or 3.2 to 8.1 or 5.5 to 10.

The embodiments illustrated as examples represent possible variants of the safety assembly 1, and it should be pointed out at this stage that the invention is not specifically limited to the variants specifically illustrated, and instead the individual variants may be used in different combinations with one another and these possible variations lie within the reach of the person skilled in this technical field given the disclosed technical teaching. Accordingly, all conceivable variants which can be obtained by combining individual details of the variants described and illustrated are possible and fall within the scope of the invention. Furthermore, individual features or combinations of features from the different embodiments illustrated and described may be construed as independent inventive solutions proposed by the invention.

Above all, the individual embodiments of the subject matter illustrated in FIGS. 1 to 5; 6 to 8; 9 constitute independent solutions proposed by the invention in their own right. The objectives and associated solutions proposed by the invention may be found in the detailed descriptions of these drawings.

### LIST OF REFERENCE NUMBERS

1 Safety assembly
2 Handling device
3 Needle assembly
4 Protective device
5 Housing chamber
6 Main part
7 Longitudinal axis
8 Distal end
9 Proximal end
10 Holding attachment
11 End wall
12 Cannula
13 Retaining part
14 Distal cannula end
15 Proximal cannula end
16 Orifice
17 Pivoting plane
18 Protective element
19 Base part
20 Locking mechanism
21 Locking element
22 Locking element
23 Passage
24 Retaining element
25 Pivot axis
26 Locking device
27 Detent element
28 Detent element
29 Bead
30 Bead
31 End face
32 Recess
33 Internal face
34 Neck
35 Spring element
36 Neck
37 External face
38 Collar

1-15. (canceled)

16. Safety assembly (1) for medical technology, in particular for taking blood samples, comprising a handling device (2) having a main part (6) of a hollow design which has a distal end (8) having a holding attachment (10) and a proximal end (9) of an open design which is designed to accommodate at least a part of a container, a longitudinal axis (7) extending between the distal end (8) and the proximal end (9), a protective device (4) having an annular base part (19) and a protective element (18) pivotally connected to the base part (19) which can be pivoted from a released position in into a guard position in order to at least partially cover a needle assembly (4) which can be attached to the handling device (2) in a guarding position, the base part (19) being retained on the holding attachment (10) of the handling device (2) so as to be rotatable about the longitudinal axis (7), a locking device (26) having at least a first detent element (27) on the handling device (2) and at least a second detent element (28) on the protective device (4), a relative position of the protective device (4) being defined with respect to the handling device (2) when the first and second detent elements (27, 28) are engaged, and the at least first detent element (27) provided on the handling device (2) is arranged spaced from the holding attachment (10) in the radial direction with respect to the longitudinal axis (7) and in the radial direction with respect to the longitudinal axis (7) on the side of the base part (19) facing away from the longitudinal axis (7), wherein the at least second detent element (28) on the base part (19) of the protective device (4) is arranged or formed on the base part (19) on the side thereof facing away from the longitudinal axis (7) and the at least second detent element (28) is disposed projecting out from the base part (19) in the radial direction, and a locking force generated between the detent elements (27, 28) when the first and second detent elements (27, 28) are in the locked position is overcome by rotation with a displacement force, and the base part (19) is thus mounted on the holding attachment (10) of the handling device (2) so as to be freely rotatable about the longitudinal axis (7).

17. Safety assembly (1) according to claim 16, wherein several first detent elements (27) are distributed circumferentially around the handling device (2).

18. Safety assembly (1) according to claim 17, wherein the first detent elements (27) are disposed or formed on a circumferentially extending neck (36) of a tubular design.

19. Safety assembly (1) according to claim 16, wherein a collar (38) projecting in the axial direction is provided on the main part (6) concentrically with the holding attachment (10) in the region of its distal end (8), which is spaced in the radial direction with respect to the holding attachment (10) apart therefrom on the side facing away from the longitudinal axis (7) and the at least first detent element (27) is disposed or formed on the collar (38).

20. Safety assembly (1) according to claim 16, wherein the main part (6) of the handling device (2) has an end wall (11) at its distal end (8) oriented in the direction perpendicular to the longitudinal axis (7) on which both the holding attachment (10) and the at least first detent element (27) are disposed or formed.

21. Safety assembly (1) according to claim 16, wherein the base part (19) of the protective device (4) has at least two diametrically opposite, slot-shaped recesses (32) on its internal face (33) facing the longitudinal axis (7), and the second detent element (28) is disposed on an external face (37) of the base part (19) facing away from the longitudinal axis (7) lying respectively opposite the recess (32).

22. Safety assembly (1) for medical technology,
in particular for taking blood samples, comprising a handling device (2) having a main part (6) of a hollow design, which has a distal end (8) having a holding attachment (10) and a proximal end (9) of an open design, which is designed to accommodate at least a part of a container, a longitudinal axis (7) extending between the distal end (8) and the proximal end (9), a protective device (4) having an annular base part (19) and a protective element (18) pivotally connected to the base part (19) which can be pivoted from a released position into a guard position in order to at least partially cover a needle assembly (4) which can be attached to the handling device (2) in the guarding position, the base part (19) being retained in the axial direction on the holding attachment (10) of the handling device (2) so as to be rotatable about the longitudinal axis (7), a locking device (26) having at least one first detent element (27) on the handling device (2) and at least one second detent element (28) on the base part (19) of the protective device (4), a relative position of the protective device (4) being defined with respect to the handling device (2) when the first and second detent elements (27, 28) are engaged, and the main part (6) of the handling device (2) has an end wall (11) at its distal end (8) oriented in the direction perpendicular to the longitudinal axis (7) on which the holding attachment (10) is disposed projecting out therefrom in the axial direction, wherein the at least first detent element (27) provided on the handling device (2) is disposed relative to the holding attachment (10) spaced from the holding attachment (10) in the radial direction with respect to the longitudinal axis (7) on the end wall (11) and projects out therefrom in the axial direction and the at least second detent element (28) provided on the base part (19) of the protective device (4) is disposed in the region of an end face (31) of the base part (19) facing the end wall (11), and wherein the first and second detent elements (27, 28) of the locking device (26) are disposed lying opposite one another in a plane, and when the first and second detent elements (27, 28) are in the locked position a locking force generated between the detent elements (27, 28) acts in the axial direction and this locking force is overcome by rotation with a displacement force and the base part (19) is thus mounted on the holding attachment (10) of the handling device (2) so as to be freely rotatable about the longitudinal axis (7).

23. Safety assembly (1) according to claim 22, wherein the at least first detent element (27) is selected from the group comprising a stud, radial tooth, projection, recess.

24. Safety assembly (1) according to claim 22, wherein several first detent elements (27) are circumferentially distributed around the end wall (11) of the handling device (2).

25. Safety assembly (1) according to claim 22, wherein the base part (19) of the protective device (4) has at least one recess (32) constituting the second detent element (28) on its end face (31) facing the end wall (11).

26. Safety assembly (1) according to claim 22, wherein the base part (19) of the protective device (4) has at least two diametrically opposite, slot-shaped recesses (32) on its internal face (33) facing the longitudinal axis (7), the recesses (32) constituting the second detent elements (28).

27. Safety assembly (1) according to claim 21, wherein it further comprises a needle assembly (3) having a cannula (12) with distal and proximal cannula ends (14, 15) spaced at a distance apart from one another in the direction of the longitudinal axis (7) and a retaining part (13) disposed on the cannula (12) between the cannula ends (14, 15) which is connected to the holding attachment (10) of the handling device (2) in the usage position, and a neck (34) of a flange-shaped design in particular is disposed on the retaining part (13) which extends at least partially out from the base part (19) in the radial direction.

28. Safety assembly (1) according to claim 27, wherein a spring element (35) is disposed between the neck (34) on the retaining part (13) of the needle assembly (3) and the base part (19) of the protective device (4).
